(19) United States
(12) Patent Application Publication
(10) Pub. No.: US 2026/0363896 A1
(43) Pub. Date: Nov. 19, 2026

(54) PAD WITH FLOATINGLY MOUNTED PAD PRESSURE ELEMENT

(71) Applicant: BAUERFEIND AG, Zeulenroda-Triebes (DE)

(72) Inventors: Dominique Panzer, Zeulenroda-Triebes (DE); Thorsten Jäger, Zeulenroda-Triebes (DE); Beatrix Bauerfeind-Johnson, Zeulenroda-Triebes (DE)

(21) Appl. No.: 19/124,831

(22) PCT Filed: Oct. 25, 2023
(86) PCT No.: PCT/EP2023/079811

§ 371 Date: Apr. 25, 2025

(30) Foreign Application Priority Data
Oct. 28, 2022 (DE) 10 2022 128 759.9

(51) Int. Cl. A61F 5/30

(52) U.S. Cl. CPC A61F 5/30

(57) ABSTRACT

The invention relates to a pad (1) for orthopedic aids, with an elastically deformable base body (3) and with at least one pad pressure element (9, 10). It is provided that the base body (3) comprises a depression (7, 8) in an outer surface (19) for the at least one pad pressure element (9, 10), the contour of which corresponding at least essentially to the contour of the pad pressure element (9, 10) such that a groove (20, 21) is formed between the base body (3) and the pad pressure element (9, 10) at least sectionally when viewed in the peripheral direction of the pad pressure element (9, 10).

The invention relates to a pad for orthopedic aids, with an elastically deformable base body and with at least one pad pressure element.

Furthermore, the invention relates to an orthosis or bandage comprising at least one such pad.

Pads that are used for various prophylactic and therapeutic applications are already known from the prior art, such as from the disclosures DE 27 22 563C2, EP 0 598 291 A1 and EP 0 600 218 A2. Furthermore, the disclosure DE 10 2019 212 740 A1 discloses a pad of the generic type. This previously known pad is characterized by peg-shaped pressure projections that are formed on a base element and are embedded together with the base element in an elastically deformable base body. This proposed solution is intended to ensure that, in addition to planar pressure, a more specific, pinpoint pressure is applied to ensure an improved massage effect. For this purpose, it is necessary that the springiness of the peg-shaped projections can be adjusted more finely than is the case in the prior art. At the same time, the new technical solution can be used in some embodiments to reduce the overall height of the pad in a positive way. This improves wearing comfort, user acceptance and, at the same time, conserves resources.

The invention is based on the task of creating a pad that, despite its advantageous massage function and effect, is less bulky and ensures a pleasant wearing comfort even during longer applications.

The task underlying the invention is solved by a pad with the features of claim 1. The pad according to the invention has the advantage that the mobility of the pad pressure element is increased by means of an adaptation that can be easily implemented constructively, and thus the massage effect remains at least the same or is improved with a softer or harder pad pressure element. According to the invention, this is achieved in that the base body comprises a depression in an outer surface for the at least one pad pressure element, the contour of which corresponds at least essentially to the outer contour of the pad pressure element such that a groove is formed between the base body and the pad pressure element at least sectionally when viewed in the peripheral direction of the pad pressure element. With the groove, a lateral distance between the base body and the pad pressure element is defined. Thus, the pad pressure element is not in direct lateral contact with the base body. The at least one pad pressure element is thus floatingly mounted in the base body. This means that during application and loading, the pad pressure element is supported laterally by the base body to a lesser extent than before and is therefore more easily deformable. This means that the pad can be dimensioned smaller in terms of height, i.e. in relation to the height or longitudinal extension of the pad pressure element, while maintaining the same massage effect. The disadvantages mentioned above are thus overcome in an advantageous way. This allows the base body to build up a certain amount of firmness due to its height and the springing behavior of the pad pressure element to be designed more flexibly. The pad is preferably configured such that the pad pressure element is located in the depression in such a way that it terminates flush with the base body, i.e. comprises a height that corresponds to the depth of the depression, protrudes beyond the base body, i.e. comprises a height that is greater than the depth of the depression, or is located in a recessed or set-back manner in the base body, i.e. comprises a height that is lower than the depth of the depression.

According to a preferred embodiment of the invention, the groove extends over the entire periphery of the pad pressure element. By forming such a peripheral groove, the contour of the depression corresponds to the (outer) contour of the pad pressure element, which on the one hand advantageously improves the mobility of the pad pressure element in all directions and on the other hand also simplifies the production of the pad.

According to an alternative embodiment of the invention, the groove extends only over one section of the periphery of the pad pressure element, and thus not over the entire periphery. This increases the mobility of one or more pressure projections only in the section. This allows the mobility and deformability of the pressure projections of a base element to be specifically influenced in order to achieve an optimal massage result. In an advantageous further development, the depression is configured such that a plurality of grooves or partial grooves, each extending over only one section of the periphery of the pad pressure element, are assigned to the pad pressure element, so that mobility is increased in these several sections and reduced in the remaining sections.

According to a preferred embodiment of the invention, the groove extends continuously or intermittently in a polygonal, circular, oval, mirror-symmetrical, asymmetrical, star-shaped and/or finger-shaped manner. The groove can ultimately comprise any geometric shape. Depending on the application, a particular shape is recommended, so that the pad can also be advantageously adapted to the application by choosing the shape of the groove. If the groove is configured continuously, i.e. without interruption, it extends in particular in a ring shape or comprises two ends in its longitudinal extension. If the groove is configured intermittently, it is divided into several partial grooves or individual grooves that follow one another in the longitudinal extension. In particular, it is provided that each partial groove is longer than the subsequent interruption in the longitudinal extension of the groove as a whole. In particular, it is provided that each partial groove is longer than the longest interruption between the partial grooves in the longitudinal extension of the groove as a whole. The groove extends preferably in a triangular, circular, rectangular, square, elliptical, kidney-shaped, hexagonal, v-shaped or w-shaped manner. In the case of a star-shaped course, it is particularly provided that the ends of the star points are not pointed but are formed rounded.

Preferably, the groove comprises a width, i.e. a distance from the base element to the base body, of at least 0.3 millimeters. It has been shown that the above-mentioned properties are achieved in a particularly advantageous way.

According to a preferred embodiment of the invention, the pad pressure element comprises a, in particular planar, base element and one or more pressure projections protruding from the base element. The base element is preferably embedded in the base body of the pad or lies in the depression of the base body. The pressure projections protrude from the base element on the side facing away from the base body, so that they can interact directly with the patient. In particular, it is provided that each pressure projection terminates flush with the base element on the side, i.e. directly on the outer edge of the base element that defines the outer contour of the pad pressure element. This means that the groove interacts directly with each pressure projection, preventing the material of the base body from directly supporting the pressure projection laterally. According to an alternative embodiment of the invention, at least one of the pressure projections is arranged laterally spaced from the outer edge or outer contour of the base element, whereby the distance of this pressure projection from the base body is further increased. If the groove extends over the entire periphery or along the entire outer contour of the pad pressure element, in particular the base element, regardless of the position or number of pressure projections on the base element, their mobility is guaranteed in the advantageous manner described above. According to a further embodiment of the invention, the pad pressure element does not comprise any pressure projection and consists, in particular, only of the planar base element. Optionally, the material of the base element is different from that of the base body, as discussed in more detail below. Due to the advantageous groove, the pad pressure element is nevertheless configured as an advantageous massage aid. Optionally, the pad is covered by a knitted fabric, at least in the area of the pad pressure element.

Preferably, one or at least one of the pressure projections, preferably each pressure projection of the at least one pad pressure element, comprises a cross-section which is many times smaller than a base area of the base element. This means that each pressure projection forms a raised area on the base element that is clearly distinguishable from the rest of the base element. In particular, this makes it possible to arrange a plurality of pressure projections on the one base element. Alternatively, the pressure projection comprises a cross-section that corresponds to or almost corresponds to the base area of the base element. As a result, the pressure projection completely or almost completely fills the base area of the base element. In this case, the pressure projection is preferably formed by a convex bulge on the free end of the pad pressure element.

According to an alternative embodiment of the invention, the pad pressure element comprises a planar base element without a pressure projection. In this case, the base element itself provides a massage function, which has an advantageous effect due to the lateral groove. In this case, the pad pressure element is preferably configured to terminate flush with the outer surface of the base body, to end set back in the depression, or to protrude outwards from the base body over the outer surface.

According to a preferred further development of the invention, the depression is configured to be so deep that the pad pressure element is completely located in the depression, wherein the one or more pressure projections terminate in particular flush with the outer surface of the base body. The depth of the depression thus corresponds at least to the height of the pad pressure element, including the pressure projections, so that the pad pressure element can be fully inserted into the depression and does not protrude beyond the outer surface of the base body. The pressure projections are recessed and can optionally also be covered by a top layer that extends over the entire base body, on the base body.

It is further provided that the pad pressure element carries only a single pressure projection. In this case, too, the depression is located directly next to the pressure projection, at least in some areas. Optionally, fasteners, such as seams or welded or adhesive connections, are provided on the outer edge of the pad pressure element.

According to an alternative embodiment of the invention, the depression is configured to be so deep, or at least one of the pressure projections is configured to be so high, that the at least one pressure projection protrudes beyond the outer surface of the base body. Preferably, the base element is completely arranged in the depression, while one or more pressure projections protrude beyond the outer surface of the base element. Particularly preferred is the depth of the depression greater than the height of the base element, such that the pressure projections lie at least sectionally within the depression. Preferably, the depression is constant over its extent, thus comprising a straight or flat bottom on which the pad pressure element rests. Alternatively, the bottom of the depression preferably deviates from a straight or constant course and varies. The shape and course of the bottom of the depression also influence the springing behavior of the pressure element in a vertical or horizontal direction. This ensures, for example, that the individual pressure projection is bent or bent over in a targeted manner in one direction during use.

According to a preferred embodiment of the invention, the pad pressure element and the base body are made of the same material. In this case, the advantageous mobility of the pressure projections is achieved by the groove according to the invention. However, using the same material reduces the manufacturing effort. In this case, the base body and base element are also preferably configured as one piece, so that it is a one-component body. This ensures a particularly time-and cost-saving production of the pad as a whole. The mobility of the pressure projections or the pad pressure element is then individually adapted to the requirement by selecting a suitable groove width and/or groove depth.

According to an alternative embodiment of the invention, the pad pressure element and the base body are made of different materials. In this variant, the mobility and thus the massage function of the pad is also adjusted by the choice of materials. In particular, the base body and/or the pad pressure element are made of a viscoelastic material that allows advantageous deformation of the pad during use, leading to the desired massage effect. In particular, the materials are chosen in such a way that they differ from each other in terms of their stiffness or elasticity.

According to a preferred first embodiment, it is provided that the pad pressure element is made of a material that is harder than the material of the base body. As a result, the pressure projections are configured to be harder than the base body and can thus transfer a higher pressure to the area to be treated at certain points. However, the mobility of the pressure projections is reduced due to the harder configuration.

According to a second embodiment of the invention, the pad pressure element is made of a material that is softer than the material of the base body, so that it comprises a higher elasticity and a reduced rigidity compared to the base body. This provides the pressure projections with a high degree of mobility, which in particular causes a chaotic movement of, in particular, several pressure projections of the pad pressure element.

In a preferred embodiment, the material of the pad pressure element has a Shore hardness of at least 40 Shore 00, preferably at least 6 Shore A and at most 20 Shore A. In a preferred embodiment, the material of the pad pressure element has a Shore hardness of at least 20 Shore A and at most 80 Shore A. In a preferred embodiment, the material of the at least one pad pressure element comprises a hardness of at least 20 Shore A and at most 60 Shore A. In a preferred embodiment, the material of the at least one pad pressure element comprises a hardness of at least 20 Shore A, more preferably at least 25 Shore A. In a preferred embodiment, the material of the at least one pad pressure element comprises a hardness of at most 50 Shore A. In a preferred embodiment, the material of the at least one pad pressure element comprises a hardness of at most 49 Shore A, more preferably of at most 45 Shore A. Preferably, the material of the at least one pad pressure element comprises a hardness of at least 35 to at most 45 Shore A. In a preferred embodiment, the material of the at least one pad pressure element comprises a hardness of about 40 Shore A. In a preferred embodiment, the material of the base body has a Shore hardness of at least 20 Shore 00 and at most 60 Shore 00 and/or the material of the pad pressure element has a Shore hardness of at least 40 Shore 00, preferably at least 6 Shore A and at most 20 Shore A.

According to a preferred further development of the invention, the groove comprises a constant depth or at least two different depths in its longitudinal extension. The depth of the groove changes or influences the height range in which the base body and the pad pressure element are spaced apart. With decreasing groove depth, the base body has a higher supporting effect on the base element and each pressure projection. With increasing depth, the influence and thus the stiffening of the pad pressure element decreases. In this way, both the width and the depth of the groove, seen in the peripheral direction of the base body, can be used to set or adapt the target function of the pad pressure element.

According to a preferred embodiment of the invention, the groove comprises a depth which corresponds to at least 10% of the height of the base body at the edge of the groove. The groove can be designed in a manner comparable to a “record groove”. Thus, it can be variably designed in terms of its width and depth.

Preferably, one or at least one of the several pressure projections is configured to be knob-shaped, peg-shaped, cylindrical, pyramidal, conical and/or mushroom-shaped. Depending on the application, a different shape is possible here. Due to the pressure projections and the design of the pad according to the invention, it is achieved that the movable pressure projections lead to an alternating pressure, as for example also exercised by a therapist during a massage, so that the pressure effect on lymph and blood vessels remains for a long time. This is particularly advantageous when the pad is configured as a knee pad, where a corresponding massage effect is applied to the meniscus base and the Hoffa's fat pads. Preferably, each pressure projection comprises a curved, in particular convex or outwardly bulging, free end face.

Preferably, one or at least one of the several pressure projections is oriented, in its longitudinal extension, perpendicular to the outer surface of the base body or inclined to the outer surface of the base body. By means of an inclined orientation of the pressure projection to the outer surface, a deformation direction is already predetermined for the pressure projection during the treatment. If pressure is applied against the inclination of the pressure projection, it appears particularly rigid and hard. If pressure is applied in the direction of the inclination, it yields all the more quickly or with all the less force. This means that, depending on the application, the pressure distribution when using the pad can be optimally adjusted for each patient. If the pressure projection extends perpendicular to the outer surface of the base body, it can be deformed equally or with the same force in all directions transverse to its longitudinal extension.

According to a preferred further development of the invention, the base element comprises a, in particular circular, base and one or more arms protruding radially from the base, wherein a pressure projection is arranged each on the base and on the end of each arm facing away from the base. The pressure projections preferably each protrude in the same direction from the base element, independently of the contour of the base element. As mentioned above, the pressure projections are preferably arranged laterally at a distance from the outer edge of the base element, so that the distance between each pressure projection and the base body or the side wall of the base body that forms the depression increases. Alternatively, the pressure projections, or at least one of the pressure projections, are arranged directly on the outer edge of the base element, so that the pressure projection laterally terminates flush with the contour or outer contour of the base element.

Due to the fact that the pressure projections are arranged at least partially on the radially extending or protruding arms of the base element, they are connected to the base in an advantageously flexible manner and the base element or pad pressure element as a whole comprises advantageous elasticity and deformability, which is particularly advantageous in the case of chaotic pressure distribution. Due to the fact that the pressure projections are each arranged at the end of one of the arms, they are surrounded by the base body, or by the groove, at least sectionally, with respect to their periphery. By arranging the pressure projections at the end of each arm, the deformation behavior of the pressure projections during application can be advantageously influenced by the groove, because the groove can extend over a wide peripheral section of the pressure element itself.

According to a preferred embodiment of the invention, the base element is configured to be star-shaped, wherein the arms are preferably distributed uniformly over the periphery of the base. In particular, the base element comprises five or six arms. The base element is particularly preferably configured to be symmetrical when viewed from above, wherein the base element can be configured to be mirror-symmetrical or point-symmetrical. Optionally, the five arms are unevenly distributed around the periphery of the base. For example, a pad pressure element comprises a base element with five arms, each of which is oriented at an equal angle to one another, wherein the angle between at least two of the arms is greater than between the remaining arms in order to achieve a greater distance between these neighboring arms. According to another embodiment, the pad pressure element comprises six arms, of which two diametrically opposed arms are each of equal length, and wherein two arms located diametrically opposite each other are longer than the other four arms. In principle, the shape of the base element can comprise any number of arms and pressure projections. For example, there can be multiple pressure projections configured to be on one arm. The base can also be configured without a pressure projection. In this case, the respective application and the area of application of the pad, as well as the position of the pressure element on the base body, are important and must be selected by a person skilled in the art correspondingly. Preferably, the pad pressure element is configured in one piece so that the base element is configured in one piece with the pressure projections.

The pad is preferably a knee pad, an ankle pad, a back pad, an elbow pad, a pad for the arm area or a pad for the abdominal area.

The orthosis or bandage according to the invention with the features of claim 22 is characterized by the pad according to the invention. This results in the advantages already mentioned above.

Preferably, the orthosis is an elastic knitted orthosis or the bandage is an elastic knitted bandage, in particular a knee bandage.

Further advantages and preferred features and combinations of features result from the above description and from the claims. The invention will be explained in more detail in the following, with reference to the drawings. For this purpose,

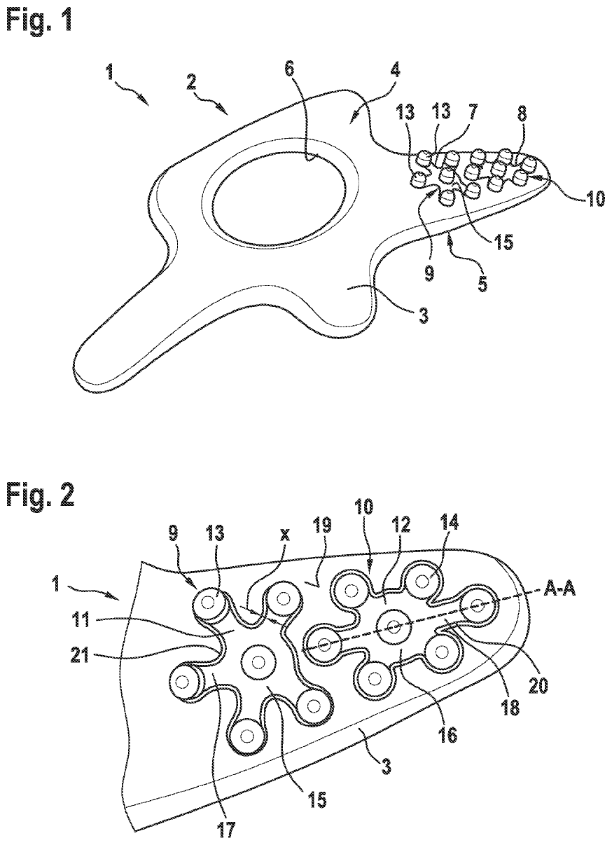

FIG. 1 shows an advantageous pad in a perspective view,

FIG. 2 shows an enlarged detailed top view of the pad from FIG. 1,

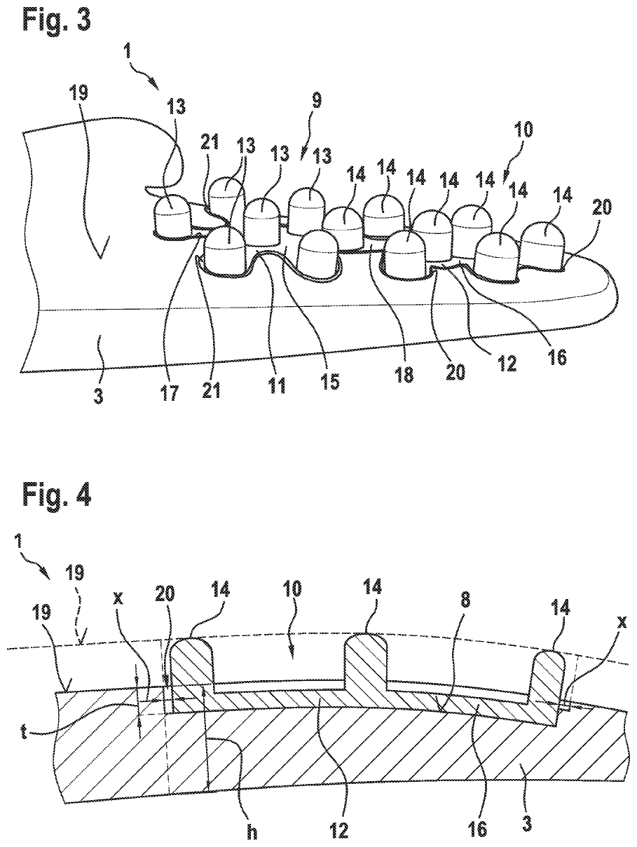

FIG. 3 shows a side view of the pad from FIG. 1,

FIG. 4 shows a simplified longitudinal section of the pad from FIG. 1,

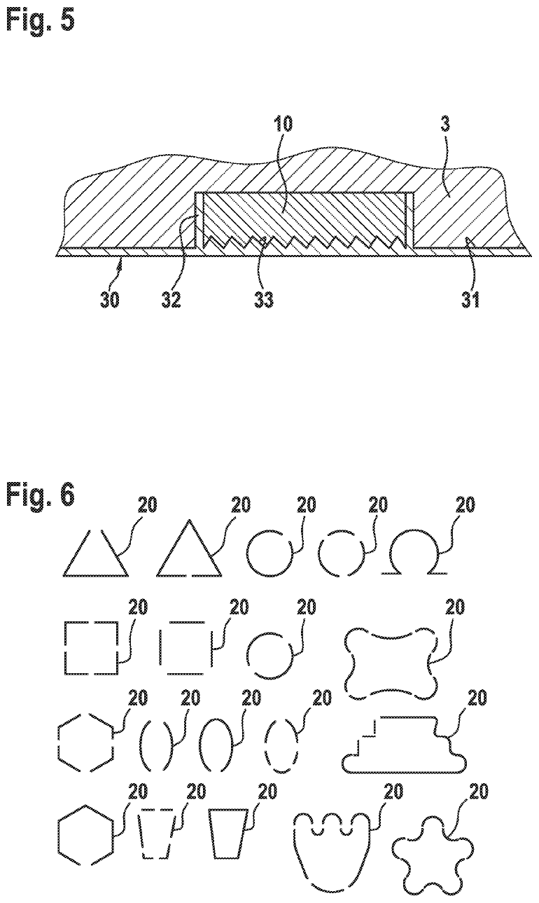

FIG. 5 shows an exemplary manufacturing method for the pad in a longitudinal section,

FIG. 6 shows exemplary groove profiles in an overview, and

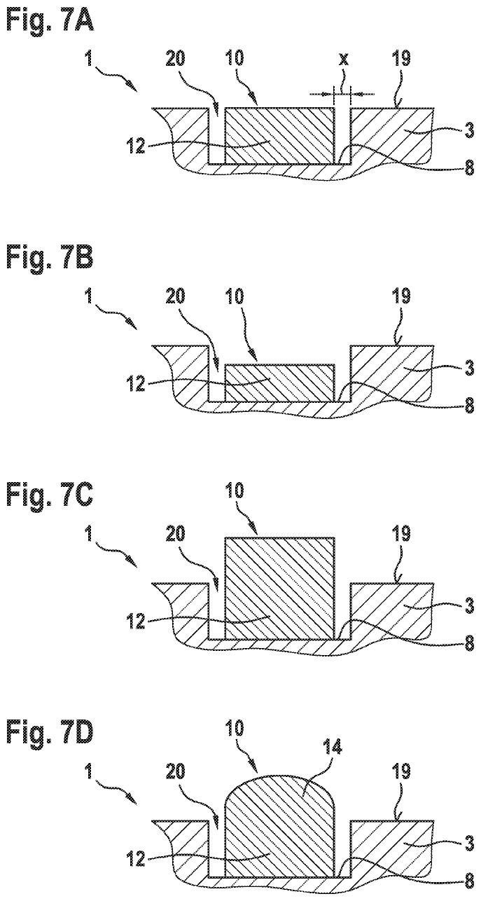

FIG. 7A to D show further embodiments of the pad, each in a simplified sectional view.

FIG. 1 shows in a perspective view an advantageous pad 1 for an orthosis or bandage 2 that is not shown in more detail here. The pad 1 has a base body 3 that is essentially configured to be planar and is made of an elastic, in particular viscoelastic, material. The base body 3 comprises a three-dimensional outer contour with a treatment side 4 and a back side 5 facing away from the treatment side 4. According to the present embodiment, the outer contour of the base body 3 is characterized by several tongues or tabs that project from a base ring, in particular radially outwards, with an oval-shaped opening 6 also configured in the base ring. However, the exact shape of the base body 3 is not important in this regard. The base body 3 comprises two depressions 7, 8 in one of the tabs, wherein a pad pressure element 9, 10 is inserted in each of the depressions. Further pad pressure elements can be arranged on the arm or on other tabs of the base body as described and configured below.

FIG. 2 shows an enlarged detailed view of pad 1 in the area of the pad pressure elements 9, 10. The pad pressure elements 9, 10 each comprise a base element 11, 12, on which there are arranged, in each case, a plurality of pressure projections 13 and 14, respectively, which protrude in the same direction from the base element 11, which is configured to be planar. Preferably, the pressure projections 13, 14 are configured in one piece with the base element 11.

The respective pad pressure element 11, 12 is also made of an elastic, preferably viscoelastic material. The base elements 11, 12 are each configured to be star-shaped in their contour, wherein each of the base elements 11, 12 comprises a circular base 15, 16 and a plurality of arms 17, 18 protruding radially from the base 15, 16. The arms 17, 18 can each be arranged uniformly or non-uniformly distributed over the periphery of the common base 15, 16. According to the present embodiment, the pad pressure element 9 comprises five arms 17 and the pad pressure element 10 comprises six arms 18. While the arms 17 of the pad pressure element 9 are each configured to be at least essentially equally long, the pad pressure element 10 has two arms 18 that are configured to be longer than the other arms 18. The longer arms 18 are located diametrically opposite each other.

The pressure projections 13, 14 of the respective pad pressure element 9, 10 are arranged in such a way that one pressure projection 13, 14 is arranged in the center of the base 15, 16 and another pressure projection 13, 14 is arranged on the respective end of the arms 17, 18 facing away from the base 15, 16. The pressure projections 13, 14 of the respective pad pressure element 9, 10 each point in the same direction or protrude in the same direction from the respective base element 11, 12, and are thus oriented parallel to one another, at least when the base elements 11, 12 are located on a flat surface. This orientation is influenced and changed by elastic deformation of the base body 3 and/or the base elements 11, 12.

The pad pressure elements 9, 10 lie within each depression 7, 8 in such a way that at least the respective base element 11, 12 is arranged countersunk in the depression 7, 8, wherein, according to the present embodiment, the pressure projections 13, 14 protrude from the outer surface 19 of the base body 3.

FIG. 3 shows the pad 1 in a side view, in which it can be easily seen that the pressure projections 13, 14 protrude beyond the outer surface 19 of the base body 3 and the base elements 11, 12 lie completely within the depression 7, 8. The depth of the depression 7,8 is greater than the height of each base element 11,12, so that the pressure projections themselves lie also partially within the depression 7,8.

For clarification, FIG. 4 shows a longitudinal section of the pad 1 in the area of the pad pressure element 10 according to the dashed line A-A drawn in FIG. 2.

The respective depression 7, 8 thus comprises a depth which, according to the present embodiment, is at least as large as the height of the inserted base element 11, 12. The pressure projections 13, 14 are configured to be of such a height that they protrude from the depression of the base body 3 of the pad 1. While the depression 8 comprises a straight or flat bottom according to the present embodiment, it is provided according to a further embodiment-not shown here-that the bottom of the depression deviates from a straight plane. This allows the behavior of the pressure projections 13, 14 to be further influenced during use and, for example, a preferred direction of bending to be specified.

The contour or inner contour of each depression 7, 8 corresponds at least essentially to the contour or outer contour of each pad pressure element 9, 10, in particular to that of each base element 11, 12. The contour of each depression 7, 8 is chosen such that that, at least in sections, as viewed in the peripheral direction of the respective base element 11, 12, a lateral distance x remains. The distance x creates a groove 20 or 21 between the respective pad pressure element 9, 10 and the base body 3, which can be seen, for example, in FIG. 2. This groove 20, 21 extends at least sectionally along the periphery of the respective pad pressure element. The pressure projections 13, 14 are each configured to be at the ends of the arms 17, 18 such that they terminate flush with the ends. Thus, each groove 20, 21 is also positioned in the area of the arms 17, 18 such that a distance between the pressure projections 13, 14 and the base body 3 is ensured. The distance x is preferably 0.1 mm to 3 mm, preferably 0.5 mm to 1.5 mm.

According to an advantageous embodiment, the respective groove 20, 21 extends over the entire periphery of the respective pad pressure element 9, 10. Alternatively, the respective groove 20, 21 extends at least sectionally over the respective periphery, wherein a plurality of such grooves 20, 21 can also be assigned to the respective pad pressure element 9, 10. The distance x along a groove 20, 21 can also change. In its longitudinal extension, each groove 20, 21 preferably comprises a constant depth t. Alternatively, the groove 20, 21 comprises a varying depth t in its longitudinal extension, as shown for example in FIG. 4, where the groove 20 in the sectional plane to the left of the pad pressure element 10 comprises a greater depth t than to the right of it.

Each groove 20, 21 preferably comprises a depth t that corresponds to at least 10% of the height h of the base body 3 at the edge of the groove 20, 21, as shown in FIG. 4. This ensures an advantageous deformation behavior of the pad pressure element 9, 10.

Due to the respective groove 20, 21, the springing behavior and also a springing direction of the respective pressure projection 13, 14 is advantageously influenced.

The pressure projections 13, 14, which in this case are configured to be in the form of knobs with rounded ends, are preferably configured to be made of a firmer material than the base body 3. However, they can also consist of a softer material. In the case of pads whose pressure projections 13, 14 are harder than the base body 3, the massage effect that can be achieved by the pad 1 or an orthosis or bandage 2 comprising a pad 1 is higher.

The depth t and width x of the respective groove 20, 21 surrounding the respective pressure projection or base element 11, 12 determines the mobility of the pressure projection 13, 14 assigned to it. Because the material of the base body 3 directly adjacent to the respective pressure projection 13, 14 is thinner or has been removed, the individual pressure projection 13, 14 can be better compressed and also move better in general within the extended edge area due to the distance x resulting from the groove 20, 21. This applies regardless of whether the respective pressure projection 13, 14 is completely countersunk into the depression 7, 8 and, for example, terminates flush with the outer surface 19 of the base body 3, or whether the respective pressure projection 13, 14 protrudes from the outer surface 19, as shown in the present embodiments.

By specifying the width and depth of the groove 20, 21 and the dimensions of the partial area surrounding the groove, i.e. the longitudinal extension of the groove, the springing behavior and the springing direction of the associated pressure projection 13, 14 can be adjusted as required. It is also conceivable that several pressure projections 13, 14 share a groove, i.e. that a groove extends between these pressure projections 13, 14. This applies regardless of whether the groove extends in a straight line or with a curve, in the form of an ellipse or a circle, as long as the pressure projections 13, 14 lie within the depression 7, 8.

While, according to the present embodiment, the pressure projections 13, 14 are configured to be knob-shaped, according to a further embodiment, the pressure projections are pyramidal, spherical, rod-shaped or peg-shaped, with a round, oval or star-shaped cross-section.

Optionally, the pressure projections 13, 14 are each configured to be equally high, so that they end in a common plane. Alternatively, the pressure projections 13, 14 are configured to be differently high, so that a profile of the respective pad pressure element 9, 10 is obtained, in a side view, which is, for example, stepped or undulating.

The higher the respective pressure projection 13, 14 is configured to be, the more mobile it becomes. According to the present embodiment, this effect is utilized in a particularly advantageous way, since the pressure projections 13, 14 protrude out of the base body 3 or its outer surface 19. Thus, during use, there is increased stimulation, which at the same time does not show a homogeneous springing behavior, but rather a chaotic effect. The respective groove 20, 21 helps to make the each pad pressure element 9, 10 smaller overall than would be the case with a flush mounting on a flat mounting surface without a groove. The pad pressure elements 9, 10 are thus floatingly mounted in the area of each groove 20, 21.

For certain applications, it may be advantageous if the pressure projections or pad pressure elements 9, 10 do not project from the base body 3, or only slightly, but are very mobile at each of their positions, in particular with respect to their pressure projections 13, 14. According to a further embodiment, the pressure projections 13, 14 thus end in particular flush with the outer surface 19, as shown in FIG. 4 by way of example by a dashed line of the outer surface.

FIG. 5 shows an example of an advantageous tool for manufacturing the pad 1. The tool 30 comprises a tool form 31 that makes it possible to manufacture both the base body 3 and the pad pressure elements 9, 10 in two steps using a casting pressure process. In FIG. 5, the pad pressure element 10 is shown in a simplified form. The tool form 31 comprises an annular projection 32, which defines the contour of the depression 7, 8 in the base body 3, and a bottom 33, the shape of which forms the pressure projections 13, 14. The bottom 33 is configured to be cup-shaped together with the projection 32, so that the material for the pad pressure element 9 or 10 is first introduced into this cup shape and then the material for the base body 3 is applied.

In order to achieve that the pad pressure elements 9, 10 comprise a higher or lower strength than the base body 3, it is also provided, in addition to the shape of the pressure projections 13, 14 and the base element 11, 12, that a different material is used for these than for the base body 3.

According to a further embodiment in FIG. 3, the pad pressure elements 9, 10 are made of the same material as the base body 3, so that the pad 1 can be produced in just one manufacturing step using the tool 3. The annular projection 32 automatically forms the groove during production, so that the above-mentioned advantages are achieved cost-effectively and with little time spent in the production of the pad 1.

FIG. 6 shows, in a simplified overview, different courses or geometries of the groove 20, which the groove 20 may comprise in its longitudinal extension or in its course, for example. The same applies, however, to the groove 21. Preferably, the groove 20 runs in each case annularly intermittently or continuously. The course of the groove 20 is shown in FIG. 6 by a line, continuous or intermittent. In the case of an intermittent course, as shown in most of the embodiments of FIG. 6, in which a total groove is formed by several grooves 20 or a groove 20 is formed by several partial grooves which follow one another in longitudinal extension, it is provided in particular that the respective interruption in the longitudinal extension of the groove 20 is in each case shorter than the partial groove of the respective groove 20, wherein each partial groove is represented by a drawn line section. According to the embodiments shown, the groove 20 may have a triangular, circular ring-shaped, elliptical, oval, omega-shaped, quadrangular, kidney-shaped, hexagonal, clamp-shaped, O-shaped, V-shaped, symmetrical or asymmetrical course, wherein the number of interruptions can be selected differently depending on the application. The groove 20 may also have a star-shaped course, wherein the star points preferably have rounded corners, wherein each star point is in particular finger-shaped. The star points are then preferably arranged evenly distributed over the periphery or asymmetrically distributed.

FIGS. 7A to 7D show further embodiments of the advantageous pad 1, each in a simplified sectional view in the area of the pad pressure element 10.

According to the embodiment shown in FIG. 7A, the pad pressure element 10 comprises only the base element 12 and is therefore configured pressure projection-free or without pressure projections. The pad pressure element 10 thus comprises, in cross-section, for example, an at least essentially rectangular or square contour. The height of the base element 12 is selected such that the pad pressure element 10 terminates flush with the outer surface 19 of the base body 3.

According to the embodiment of FIG. 7B, the height of the pressure projection-free pad pressure element 10 or of the base element 12 is selected such that it is smaller than the depth of the depression 8, whereby the pad pressure element 10 lies completely in the depression 8 and ends set back relative to the outer surface 19.

According to the embodiment of FIG. 7C, it is provided that the pad pressure element 10 or the base element 12 without pressure projection already comprises a height which is greater than the depth of the depression 8, so that the pad pressure element 10 itself forms a projection which protrudes from the outer surface 19 of the base body 3.

According to the embodiment shown in FIG. 7D, the pad pressure element 10 comprises a single pressure projection 14, which is formed by the in this case convexly shaped or bulged free end face, i.e. the end face of the pad pressure element 10 facing away from the base body 3. The bulge extends over the entire end face of the base element 12, resulting in a pressure projection 14 whose outer contour almost or exactly corresponds to the outer contour of the base element 12.

The pressure projection(s) (14) of the pad pressure element (10) thus has, as shown in the embodiment of FIGS. 1 to 4, preferably a cross-sectional area which is significantly smaller than that of the associated base element (12), or which preferably corresponds to or almost corresponds to the base area of the base element (12), as shown, for example, in the embodiment of FIG. 7D.

1. A pad for orthopedic aids, with an elastically deformable base body (3) and with at least one pad pressure element (9, 10), characterized in that the base body (3) comprises a depression (7, 8) in an outer surface (19) for the at least one pad pressure element (9, 10), the contour of which corresponding at least essentially to the contour of the pad pressure element (9, 10) such that a groove (20, 21) is formed between the base body (3) and the pad pressure element (9, 10) at least sectionally when viewed in the peripheral direction of the pad pressure element (9, 10).

2. The pad according to claim 1, characterized in that the groove (20, 21) extends over the entire periphery of the pad pressure element (9, 10).

3. The pad according to claim 1, characterized in that the groove (20, 21) extends over only one section of the periphery of the pad pressure element (9, 10).

4. The pad according to claim 1, characterized in that the groove (20, 21) extends continuously or intermittently in a polygonal, circular, oval, mirror-symmetrical, asymmetrical, star-shaped or finger-shaped manner.

5. The pad according to claim 1, characterized in that the groove (20, 21) comprises a width of at least 0.3 between the pad pressure element (9, 10) and the base body (3).

6. The pad according to claim 1, characterized in that the pad pressure element (9, 10) comprises a, in particular planar, base element (11, 12) and one or more pressure projections (13, 14) protruding from the base element (11, 12).

7. The pad according to claim 1, characterized in that at least one pressure projection (13, 14) comprises a cross-section which is many times smaller than a base area of the base element (11, 12) or corresponds to or almost corresponds to the base area of the base element (11, 12).

8. The pad according to claim 1, characterized in that the pad pressure element (9, 10) comprises a planar base element (11, 12) without a pressure projection.

9. The pad according to claim 1, characterized in that the depression (7, 8) is configured to be so deep that the pad pressure element (9, 10) is completely located in the depression (7, 8), wherein in particular at least one pressure projection (13, 14) terminates flush with the outer surface (19) of the base body (3).

10. The pad according to claim 1, characterized in that at least one pressure projection (13, 14) protrudes beyond the outer surface (19) of the base body (3).

11. The pad according to claim 1, characterized in that the at least one pad pressure element (9, 10) and the base body (3) are made of the same material.

12. The pad according to claim 1, characterized in that the at least one pad pressure element (9, 10) and the base body (3) are made of different materials.

13. The pad according to claim 1, characterized in that the at least one pad pressure element (9, 10) is made of a harder or softer material than the base body (3).

14. The pad according to claim 1, characterized in that the groove (20, 21) comprises a constant depth (t) or at least two different depths (t) in its longitudinal extension.

15. The pad according to claim 1, characterized in that the groove (20, 21) comprises a depth (t) which corresponds to at least 10% of the height (h) of the base body (3).

16. The pad according to claim 1, characterized in that at least one pressure projection (13, 14) is configured to be knob-shaped, peg-shaped, cylindrical, pyramidal, conical and/or mushroom-shaped.

17. The pad according to claim 1, characterized in that at least one pressure projection (13, 14) is oriented, in its longitudinal extension, perpendicular to the outer surface (19) of the base body (3) or inclined to the outer surface (19) of the base body (3).

18. The pad according to claim 1, characterized in that the base element (3) comprises a, in particular circular, base (15, 16) and one or more arms (17, 18) protruding radially from the base (15, 16), wherein at least one pressure projection (13, 14) is arranged each on the base (15, 16) and on that end of each arm (17, 18) facing away from the base (15, 16).

19. The pad according to claim 1, characterized in that the base element (11, 12) is configured to be star-shaped.

20. The pad according to claim 1, characterized in that at least two of the arms (17, 18) are configured to be the same length or different lengths.

21. The pad according to claim 1, characterized in that the pad (1) is a knee pad, an ankle pad, a back pad, an elbow pad, a pad for the arm area or a pad for the abdominal area.

22. An orthosis or bandage (2) comprising a pad (1) according to claim 1.
